(19) United States
(12) Patent Application Publication
(10) Pub. No.: US 2008/0177290 A1
(43) Pub. Date: Jul. 24, 2008

(54) Bone clamp assembly

(75) Inventors: Rebecca Hawkins Wahl, Escondido, CA (US); Ernie Corrao, Bethel, CT (US); Alan Taylor, Memphis, TN (US)

Correspondence Address: Roeder & Broder LLP, 5560 Chelsea Avenue, La Jolla, CA, 92037, US

(73) Assignee: Nexa Orthopedics, Inc.

(21) Appl. No.: 11/655,713

(22) Filed: Jan. 18, 2007

(51) Int. Cl. A61B 17/08

(52) U.S. Cl. 606/151

(57) ABSTRACT

A clamp assembly (16) for stabilizing one or more adjacent regions (12A) (12B) and for selecting a bone screw (18) to be inserted into the one or more adjacent regions (12A) (12B) includes a first clamp side (224), a second clamp side (226) and a connector (228) that pivotally secures the first clamp side (224) and the second clamp side (226) together. The first clamp side (224) includes a first locking component (236) having a first plurality of first teeth (236B) and the second clamp side (226) includes a second locking component (244) having a second plurality of second teeth (244B). The plurality of first teeth (236B) selectively engage the plurality of second teeth (244B) when the clamp assembly (16) is positioned about the bone regions (12A) (12B) so that the clamp assembly (14) is in a locked position with the first clamp side (224) and the second clamp side (226) engaging substantially opposite sides of the adjacent regions (12A) (12B). Additionally, the clamp assembly (16) includes a screw guide (20) that is secured to at least one of the clamp sides (224) (226). The screw guide (20) provides information for selecting the appropriate bone screw (18) based on the relative position of the clamp sides (224) (226).

### BACKGROUND

It is often necessary to fuse two bone regions to repair a fracture or to fuse a joint. One type of device used to fuse two bone regions is a bone screw. Because the size and orientation of the bone regions that need to be fused can vary greatly, the bone screws are designed in varying lengths and diameters. For any particular bone fusing procedure, the size of the bone screw must be chosen properly so as to maximize the stability of the bone regions after they are fused together, and to minimize the irritation adjacent to the area where the bone screw has been inserted.

### SUMMARY

The present invention is directed toward a clamp assembly for clamping adjacent regions, e.g. bone regions, together so that a bone screw can be successfully inserted into the adjacent regions. The clamp assembly includes a first clamp side, a second clamp side, and a screw guide that is secured to at least one of the clamp sides. The first clamp side includes a first engager that engages a first area of one of the adjacent regions. The second clamp side includes a second engager that engages a second area of one of the adjacent regions. The screw guide provides information for selecting the appropriate bone screw based on the relative position of the clamp sides. With this design, the physician using the clamp assembly can easily and accurately determine the appropriate bone screw to be inserted into the adjacent regions.

In one embodiment, the first clamp side includes a first locking component and the second clamp side includes a second locking component that interacts with the first locking component to lock the clamp sides together in a locked position. Further, the screw guide can be secured to at least one of the locking components. With this design, the locking components can provide information for selecting the bone screw when the locking components are in the locked position, and the clamp assembly holds and stabilizes the bone regions while the bone screw is inserted into the bone regions. By enabling the physician to select the appropriate size bone screw based on information provided on the clamp assembly when the clamp assembly is in the locked position on the adjacent regions, the size of the bone screw is accurately determined, and any irritation to the bone regions and to the tissue adjacent to the bone screw will be minimized. As a result thereof, the likelihood of success of the procedure is enhanced.

In one embodiment, the first locking component includes a plurality of first teeth and the second locking component includes a plurality of second teeth that selectively engage the first teeth.

Additionally, the clamp assembly can include a connector that pivotally secures the clamp sides together and allows for relative rotation of the clamp sides.

In one embodiment, the first engager includes a first engagement surface that inhibits relative movement between the first engager and the adjacent regions and the second engager includes a second engagement surface that inhibits relative movement between the second engager and the adjacent regions.

The present invention is further directed to a bone fusion assembly including a bone screw and the clamp assembly that holds and stabilizes the bone regions while the bone screw is inserted into the bone regions.

The present invention is also directed a method for selecting a bone screw that is inserted into one or more bone regions. The method includes the steps of providing a first clamp side having a first engager that engages a first area, providing a second clamp side having a second engager that engages a second area, and provides information for selecting the appropriate bone screw based on the relative position of the clamp sides with a screw guide.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified side view of a pair of adjacent regions, in cut-away, that are fused together, and a bone fusion assembly having features of the present invention;

FIG. 2A is a perspective view of a clamp assembly having features of the present invention;

FIGS. 2B and 2C are alternative side views of the clamp assembly of FIG. 2A;

FIG. 2D yet another side view of the clamp assembly of FIG. 2A;

FIG. 2E is a top view of the clamp assembly of FIG. 2A;

FIG. 2F is an end view of the clamp assembly of FIG. 2A;

FIG. 2G is a cut-away view taken on line 2G-2G in FIG. 2B;

FIGS. 3A and 3B are alternative views of a first clamp side having features of the present invention;

FIG. 3C is a cut-away view taken on line 3C-3C of FIG. 3A;

FIGS. 4A and 4B are alternative views of a second clamp side having features of the present invention; and

FIG. 4C is a cut-away view taken on line 4C-4C of FIG. 4A.

### DESCRIPTION

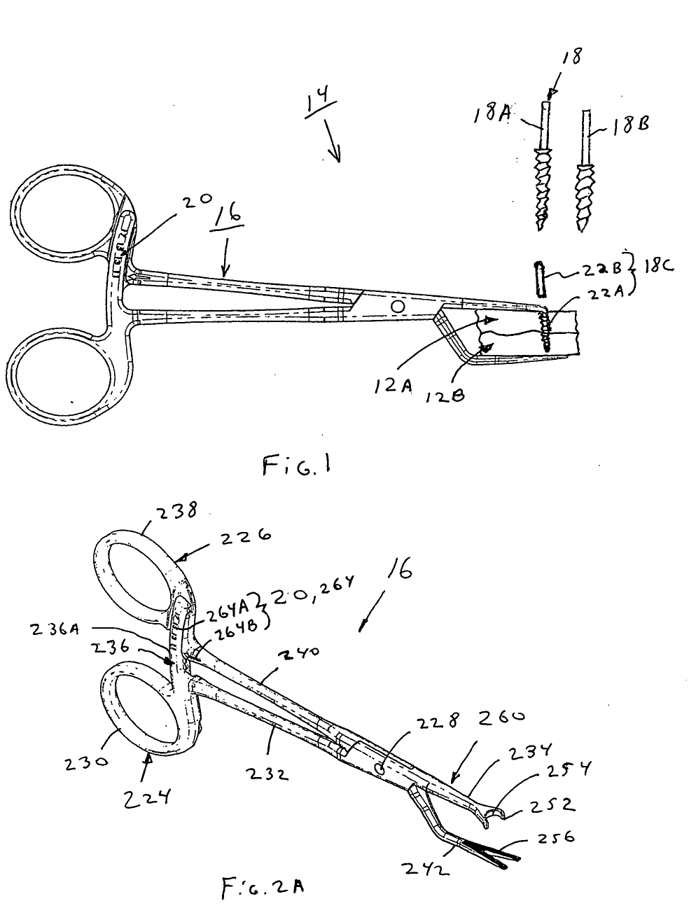

FIG. 1 is a simplified side view of a first region 12A and an adjacent second region 12B, and a bone fusion assembly 14 having features of the present invention. As illustrated herein, the bone fusion assembly 14 includes a clamp assembly 16 and one or more bone screws 18 that are used to fuse the adjacent regions 12A, 12B together. The clamp assembly 16 holds the adjacent regions 12A, 12B together and stabilizes the adjacent regions 12A, 12B while one of the bone screws 18 is inserted into the adjacent regions 12A, 12B. The bone screw 18 urges the adjacent regions 12A, 12B together so that the adjacent regions 12A, 12B are fused together.

The type and location of the adjacent regions 12A, 12B urged together can vary. For example, each region 12A, 12B can be a bone or a portion of a bone of a human or other being, and the bone screw 18 can be used to fuse a fracture of a human bone, to immobilize and fuse a human joint, or to fuse together adjacent bones. Alternatively, for example, one or more bone screws 18 can be used to secure a plate (not shown) or other device to one or more bone regions 12A, 12B.

As an overview, in certain embodiments, the physician is provided with a plurality of alternatively sized bone screws 18, and the clamp assembly 16 includes a screw guide 20 that allows the physician to select the proper sized bone screw 18 for that particular procedure. For example, the physician can be provided with two or more bone screws 18 of different length. In FIG. 1, three bone screws 18A, 18B, 18C are illustrated. In this embodiment, the first bone screw 18A is the longest, the second bone screw 18B is next longest, and the third bone screw 18C is the shortest. Alternatively, the physician can be provided with more than three or less than three bone screws 18.

In FIG. 1, the third bone screw 18C has been inserted into the bone regions 12A, 12B and fuses the bone regions 12A, 12B together. Further, as illustrated in FIG. 1, the third bone screw 18C is a snap-off type screw. In this embodiment, the third bone screw 18C includes a threaded region 22A and a driver region 22B. The threaded region 22A is threaded into one or more of the bone regions 12A, 12B, and the driver region 22B is used to rotate the threaded region 22A during insertion. For example, the driver region 22B can be engaged by an insertion tool (not shown) such as a wire driver, a drill, or a screwdriver, to facilitate rotation of the bone screw 18C and insertion of the threaded region 22A into the one or more bone regions 12A, 12B. The design and size of each region 22A, 22B can vary according to the desired use for the bone screw 18C. With a snap-off screw, the driver region 22B is designed to break way from the threaded region 22A after the threaded region 22A is inserted into the bone regions 12A, 12B. In FIG. 1, the third bone screw 18C is illustrated in a decoupled configuration in which the driver region 22A has been separated from the threaded region 22A.

Alternatively, one or more of the bone screws 18 can be another type of screw.

It should be noted that the bone screw 18 can be inserted with a pilot hole (not shown) or without a pilot hole, depending upon the condition and type of the one or more bone regions 12A, 12B. In one embodiment, the bone screw 18 is self drilling and/or tapping.

The clamp assembly 16 fixedly clamps to at least one of the bone regions 12A, 12B, and the screw guide 19 provides guidance that allows the physician to select the proper sized bone screw 18 for that particular procedure. In FIG. 1, the clamp assembly 16 fixedly urges the bone regions 12A, 12B together. Further, with the bone regions 12A, 12B urged together, the physician can use to the screw guide 20 to quickly determine the correct bone screw 18 to use for that procedure. Because the proper length of bone screw 18 is selected for that procedure, the desired amount of threaded region 22A is positioned in the bone regions 12A, 12B to maximize stability of the fusion, and the tip of the threaded region 22A is less likely to extend through the bottom of the bone regions 12A, 12B. If the threaded region 22A extended through the bottom, this can provide an area of irritation for soft tissue (not shown) around the inserted bone screw 18. Thus, with the desired amount of threaded region 22A in the bone regions 12A, 12B and no protrusion, the likelihood of success of the procedure is significantly enhanced.

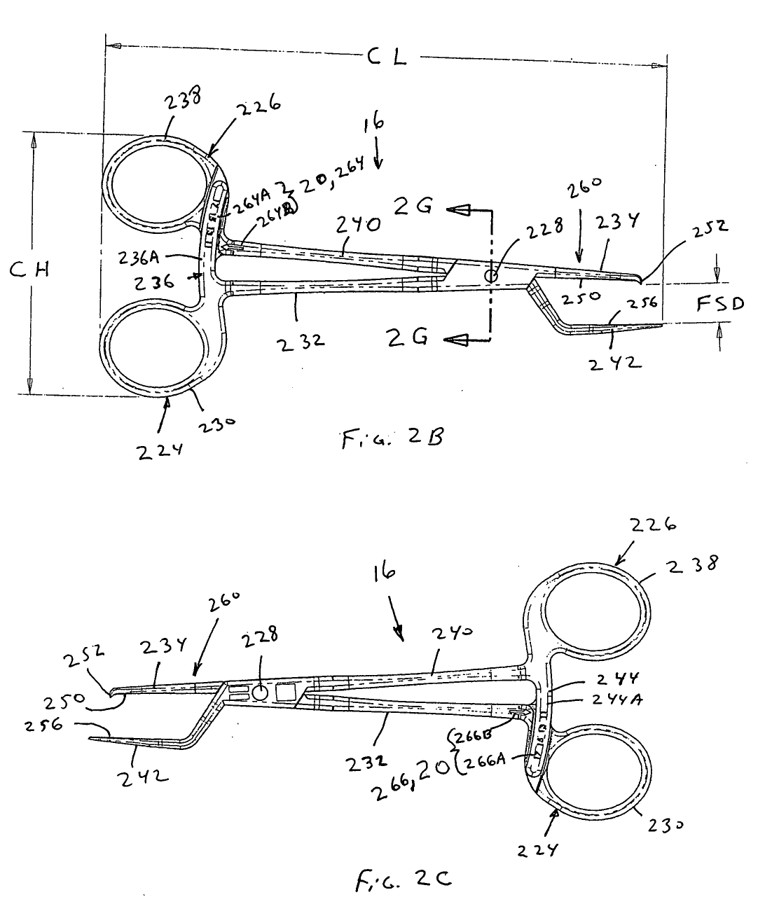

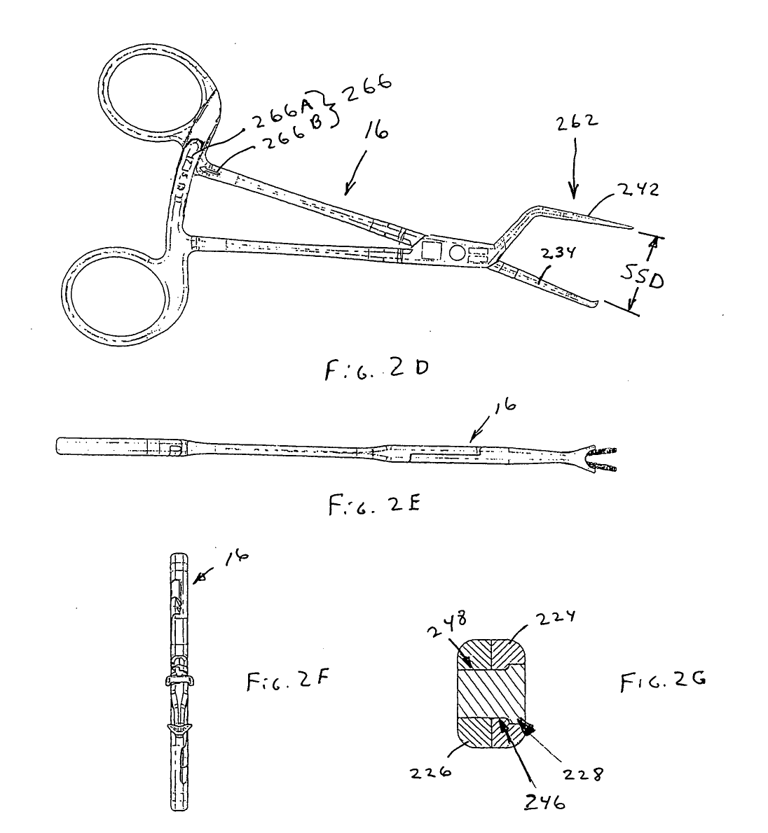

FIG. 2A is a perspective view, and FIGS. 2B and 2C are alternative side views of the clamp assembly 16. In this embodiment, the clamp assembly 16 includes a first clamp side 224, a second clamp side 226, a connector 228 that pivotally secures the clamp sides 224, 226 together, and the screw guide 20. The design of each of these components can be varied pursuant to the teachings provided herein. In one embodiment, the first clamp side 224 and the second clamp side 226 pivot relative to each other about the connector 228 in a scissors-like fashion.

The first clamp side 224 includes a first handle 230, a first shaft 232, a first engager 234, and a first locking component 236. Similarly, the second clamp side 226 includes a second handle 238, a second shaft 240, a second engager 242, and a second locking component 244.

The first handle 230 and the second handle 238 are utilized by the user to hold the clamp assembly 16 and to manipulate the first engager 234 and the second engager 242 around the bone regions 12A, 12B (illustrated in FIG. 1). In one embodiment, each of the handles 230, 238 is a generally annular shaped ring that is attached to a distal end of the respective shaft 232, 240. Alternatively, the handles 230, 238 can have another shape or configuration. The openings of the handles 230, 238 are adapted to receive a thumb and/or a finger of the user, so as to enable the user to effectively manipulate the clamp assembly 16.

The first shaft 232 extends between the first handle 230 and the first engager 234. Located along the first shaft 232 is a first shaft aperture 246 (illustrated in FIG. 2G) which is adapted to receive the connector 228. In the embodiment illustrated in the Figures, the first shaft aperture 246 is positioned somewhat closer to the first engager 234 than to the first handle 230. Alternatively, the first shaft aperture 246 can be positioned approximately half-way between the first handle 230 and the first engager 234 or the first shaft aperture 246 can be positioned somewhat closer to the first handle 230 than to the first engager 234.

Similarly, the second shaft 240 extends between the second handle 238 and the second engager 242. Located along the second shaft 240 is a second shaft aperture 248 (illustrated in FIG. 2G), which is adapted to receive the connector 228. In the embodiment illustrated in the Figures, the second shaft aperture 248 is positioned somewhat closer to the second engager 242 than to the second handle 238. Alternatively, the second shaft aperture 248 can be positioned approximately half-way between the second handle 238 and the second engager 242 or the second shaft aperture 248 can be positioned somewhat closer to the second handle 238 than to the second engager 242. The first shaft aperture 246 and the second shaft aperture 248 are positioned so as to allow the connector 228 to extend through both of the shaft apertures 246, 248 simultaneously, thereby enabling the first clamp side 224 and the second clamp side 226 to rotate relative to each other in a scissors-like fashion.

The design of each engager 234, 236 can be varied pursuant to the type of procedure being performed. In one embodiment, the first engager 234 is located at the proximal end of the first shaft 232 and includes a first engagement surface 250 that engages a first area of the bone regions 12A, 12B, and a pair of spaced apart tips 252 that cantilever downward toward the second bone engager 242 and that engage the first area of the bone regions 12A, 12B. With this design, the first engager 234 is able to effectively and securely engage the first area of the bone regions 12A, 12B.

Additionally, the first engager 234 can include substantially, semi-circular shaped guide surface 254 positioned between the tips 252. The guide surface 254 provides a guide for inserting the bone screw 18 (illustrated in FIG. 1) into the bone regions 12A, 12B.

In one embodiment, the first engagement surface 250 can be roughened so that the first engagement surface 250 has a high coefficient of friction. Alternatively, for example, the first engagement surface 250 can be coated with a material having a high coefficient of friction. With either design, the first engagement surface 250 will be able to effectively inhibit relative movement between the first engager 234 and the first area of the bone regions 12A, 12B.

The second engager 242 engages a second area of the bone regions 12A, 12B substantially opposite to the first area of the bone regions 12A, 12B that is engaged by the first engager 234. In one embodiment, the second engager 242 is located at the proximal end of the second shaft 240 and includes a second engagement surface 256 can be substantially flat. Additionally, the end of the second engagement surface 256 can be somewhat V-shaped.

In one embodiment, the second engagement surface 256 can be roughened so that the second engagement surface 256 has a high coefficient of friction. Alternatively, for example, the second engagement surface 256 can be coated with a material having a high coefficient of friction. With either design, the second engagement surface 256 will be able to effectively inhibit relative movement between the second bone engager 242 and the second area of the bone regions 12A, 12B.

Accordingly, working together the first engager 234 and the second engager 242 are designed to immobilize and stabilize the bone regions 12A, 12B so as to allow for relatively easy and effective insertion of the bone screw 18 into the bone regions 12A, 12B.

The first locking component 236 of the first clamp side 224 and the second locking component 244 of the second clamp side 226 are designed to selectively engage each other so as to enable the clamp assembly 16 to be effectively locked when the clamp assembly 16 is positioned about the bone regions 12A, 12B. In one embodiment, the locking components 236, 238 are able to lock at a number of alternative positions depending upon the size of the bone regions 12A, 12B. FIGS. 2A-2C illustrate the clamp assembly 16 in a first locked position 260 while FIG. 2D illustrates the clamp assembly 16 in a second locked position 262 that is different than the first locked position 260. In the first locked position 260, the engagers 234, 242 are spaced apart a first separation distance FSD; and in the second locked position 262, the engagers 234, 242 are spaced apart a second separation distance SSD that is greater than the first separation distance FSD. In one non-exclusive example, the clamp assembly 16 can be adjustable for separation distances of between approximately 11 and 17 millimeters.

It should be noted that the locked positions 260, 262 illustrated in the Figures are merely representative of any number of possible locked positions 260, 262.

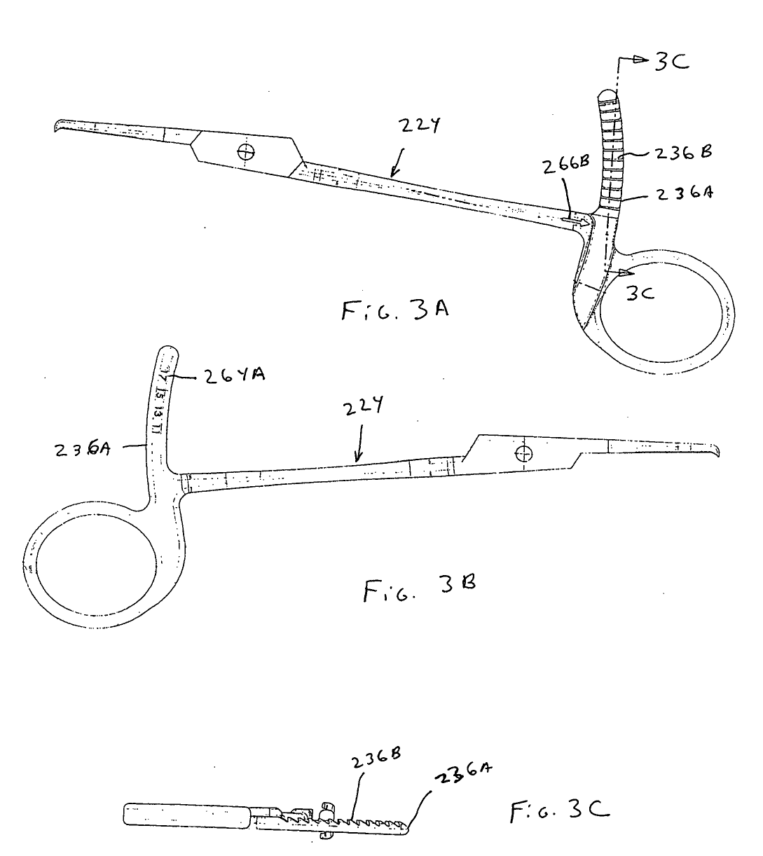

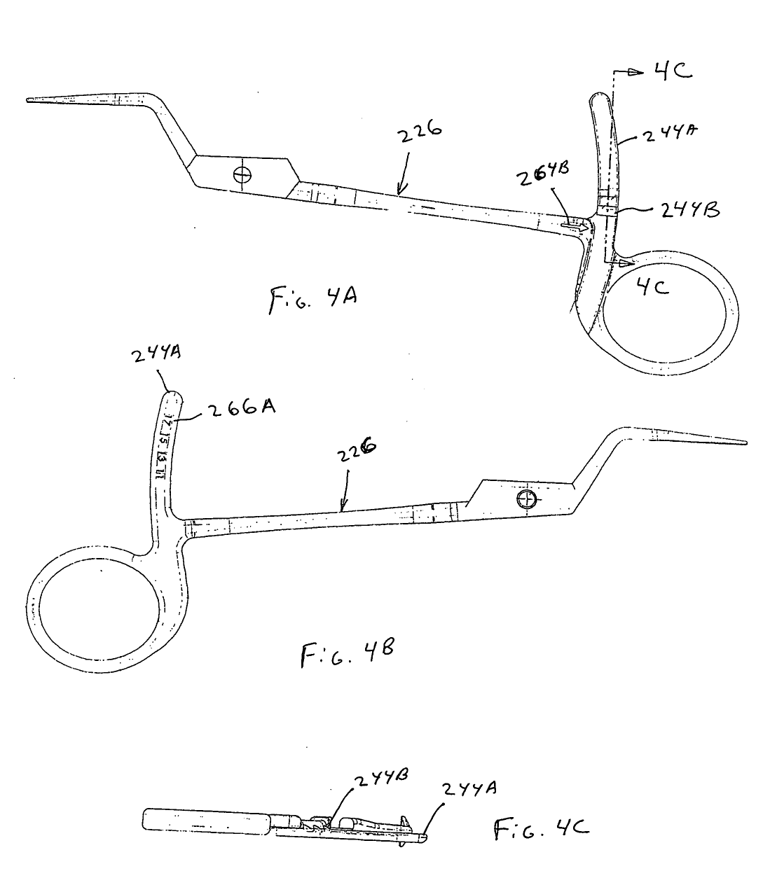

The design of each of the locking components 236, 244 can vary. In one embodiment, the first locking component 236 includes a first curved beam 236A that cantilevers away from the first shaft 232. In this embodiment, the first curved beam 236 includes a plurality of first teeth 236B (illustrated in FIG. 3A). Similarly, the second locking component 244 includes a second curved beam 244A that cantilevers away from the second shaft 240. Further, in this embodiment, the second curved beam 244A includes a plurality of second teeth 244B (illustrated in FIG. 4A).

In the Figures, the first teeth 236B are urged against the second teeth 244B. With this design, one or more of the first teeth 236A engage one or more of the second teeth 244B to selectively lock the clamp assembly 16.

Alternatively, the clamp assembly 16 can be selectively locked in a different fashion.

The screw guide 20 provides information that allows the physician to select the proper sized bone screw 18 for that particular procedure. The design of the screw guide 20 can vary. In the embodiment illustrated in the Figures, the screw guide 20 is fixedly secured to and incorporated into the clamp sides 224, 226. In this embodiment, the screw guide 20 actually includes a first side guide 264 (illustrated in FIGS. 2A and 2B) that is visible to the physician when looking at one side of the clamp assembly 16 and a second side guide 266 (illustrated in FIG. 2C) that is visible to the physician when looking at the other side of the clamp assembly 16.

In FIG. 2B, the first side guide 264 includes a plurality of spaced apart first identifiers 264A that are incorporated into the front surface of the first curved beam 236A and a first indicator 264B that is incorporated into the second shaft 240 near where the second curved beam 244A extends away from the second shaft 240. Similarly, in FIG. 2C, the second side guide 266 includes a plurality of spaced apart second identifiers 266A that are incorporated into the front surface of the second curved beam 244A and a second indicator 266B that is incorporated into the first shaft 232 near where the first curved beam 236A extends away from the first shaft 232. With this design, relative movement of the clamp sides 224, 226 results in relative movement between the first identifiers 264A and the first indicator 264B and relative movement between the second identifiers 266A and the second indicator 266B.

For example, each of the identifiers 264A, 266A can be a number, letter or other symbol that corresponds to a particular sized bone screw 18. In the Figures, each of the identifiers 264A, 266A is a number. For example, the numbers “17”, “15”, “13”, and “11” are illustrated. Alternatively, more than four or fewer than four numbers can be utilized.

Further, for example, each of the indicators 264B, 266B can be an arrow or other type of character that can point to the respective identifiers 264A, 266A.

Referring to FIGS. 2B and 2C, with the clamp assembly 16 in the first locked position 260, both side guides 264, 266 illustrate that the bone screw 18 that corresponds to “11” is the appropriate size to be inserted into the bone regions 12A, 12B. In this embodiment, each of the indicators 264B, 266B is aligned with the respective “11” identifier 264A, 266B.

In contrast, in FIG. 2D, the clamp assembly 16 is illustrated in the second locked position 262. In this position, the second side guide 266 illustrates that the bone screw 18 that corresponds to “17” is the appropriate size to be inserted into the bone regions 12A, 12B. In this embodiment, the second indicator 266B is aligned with the “17” identifier 266A.

Referring back to FIG. 2B, in one non-exclusive embodiment, the clamp assembly 16 has a clamp length “CL” of approximately 5.7 inches, and a clamp height “CH” of approximately 2.7 inches. However, other sizes can be utilized. The clamp sides 224, 226 can be made of rigid material such as stainless steel or titanium.

FIG. 2E is a top view and FIG. 2D is an end view of the clamp assembly 16.

FIG. 2G is a cut-away view taken on line 2G-2G in FIG. 2B. FIG. 2G illustrates the first shaft aperture 246 and the second shaft aperture 248 are positioned so as to allow the connector 228, e.g. a pin to extend through both of the shaft apertures 246, 248 simultaneously, thereby enabling the first clamp side 224 and the second clamp side 226 to rotate relative to each other. In one embodiment, the first shaft aperture 246 is a free fit with the connector 228 and the second shaft aperture 248 is an interference fit with the connector 228.

FIGS. 3A and 3B illustrate alternative side views of the first clamp side 224, and FIG. 3C illustrates a cut-away view taken on line 3C-3C in FIG. 3A. These Figures illustrate the first curved beam 236A, the first teeth 236B, the first identifiers 264A, and the second indicator 266B.

FIGS. 4A and 4B illustrate alternative side views of the second clamp side 226, and FIG. 4C illustrates a cut-away view taken on line 4C-4C in FIG. 4A. These Figures illustrate the second curved beam 244A, the second teeth 244B, the second identifiers 266A, and the first indicator 264B.

While the particular bone fusion assembly 14 as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A clamp assembly for engaging one or more adjacent regions for a fusion procedure that utilizes a bone screw that is inserted into the one or more adjacent regions, the clamp assembly comprising:
a first clamp side including a first engager that engages a first area of one of the adjacent regions;
a second clamp side including a second engager that engages a second area of one of the adjacent regions, the clamp sides cooperating to urges the first and second areas together; and
a screw guide that provides information for selecting the proper bone screw based on the relative position of the clamp sides, the screw guide being secured to at least one of the clamp sides.

2. The clamp assembly of claim 1, wherein the first clamp side includes a first locking component and the second clamp side includes a second locking component that interacts with the first locking component to lock the clamp sides together in a locked position.

3. The clamp assembly of claim 2 wherein the screw guide is positioned near at least one of the locking components.

4. The clamp assembly of claim 2 wherein the first locking component including a plurality of first teeth and the second locking component includes a plurality of second teeth that interact with the first teeth to lock the clamp sides together in the locked position.

5. The clamp assembly of claim 1 further comprising a connector that pivotally secures the first clamp side and the second clamp side together.

6. The clamp assembly of claim 1 wherein the first engager includes a first engagement surface that engages the first area, wherein the first engagement surface inhibits relative movement between the first engager and the first area.

7. The clamp assembly of claim 1 wherein the first engager includes a guide surface that guides the bone screw during insertion of the bone screw into the adjacent regions.

8. The clamp assembly of claim 1 wherein the screw guide includes a plurality of first identifiers that are secured to and move with the first clamp side, and a first indicator that is secured to and moves with the second clamp side.

9. A bone fusion assembly for fusing one or more adjacent, the bone fusion assembly comprising a plurality of bone screws and the clamp assembly of claim 1.

10. A bone fusion assembly for fusing one or more adjacent, bone regions together, the bone fusion assembly comprising:
a plurality of bone screws that are adapted to be inserted into the bone regions; and
a clamp assembly that holds and stabilizes the bone regions, wherein the clamp assembly includes a screw guide that provides information for selecting the proper bone screw to be inserted into the bone regions.

11. The bone fusion assembly of claim 10 wherein the clamp assembly includes a first clamp side and a second clamp side and wherein the clamp sides can be locked to hold and stabilize the bone regions.

12. The bone fusion assembly of claim 11 wherein the first clamp side includes a first engager that engages a first area of one of the bone regions, and the second clamp side includes a second engager that engages a second area of one of the bone regions, and wherein the first engager includes a guide surface that guides the bone screw during insertion of the bone screw into the bone regions.

13. The clamp assembly of claim 11 wherein the screw guide includes a plurality of first identifiers that are secured to and move with the first clamp side, and a first indicator that is secured to and moves with the second clamp side.

14. A method of selecting a bone screw that is inserted into one or more adjacent regions, the method comprising the steps of:
engaging a first area of one of the adjacent regions with a first clamp side;
engaging a second area of one of the adjacent regions with a second clamp side, the clamp sides cooperating to urges the first and second areas together; and
providing information for selecting the proper bone screw based on the relative position of the clamp sides with a screw guide, the screw guide being secured to at least one of the clamp sides.

15. The method of claim 14 further comprising the step of locking the clamp sides with the clamp sides urging the adjacent regions together.

16. The method of claim 14 further comprising the step of pivotable connecting the clamp sides together with a connector.

17. The method of claim 14 further comprising the step of guiding the bone screw with a guide surface of the first clamp side.

18. The method of claim 14 wherein the step of providing information includes securing a plurality of first identifiers to the first clamp side, and securing a first indicator to the second clamp side.
